(19) United States
(12) Patent Application Publication
(10) Pub. No.: US 2022/0031138 A1
(43) Pub. Date: Feb. 3, 2022

(54) Multifunctional Mopping Machine

(71) Applicant: Ningbo Foura Electronics Co., Ltd., Ningbo (CN)

(72) Inventor: Shizhong Lou, Ningbo (CN)

(21) Appl. No.: 17/371,346

(22) Filed: Jul. 9, 2021

(30) Foreign Application Priority Data
Aug. 3, 2020 (CN) 202021585797.3

(51) Int. Cl. A47L 11/30; A47L 11/40; C02F 1/461; C02F 1/32; C25B 1/26; C25B 9/00; A61L 2/10

(52) U.S. Cl. CPC A47L 11/305; A61L 2202/17; A47L 11/4075; A47L 11/4005; A47L 11/4041; A47L 11/4027; A47L 11/4069; C02F 1/46104; C02F 1/325; A47L 11/405; A47L 11/4088; A47L 11/4091; C25B 1/26; C25B 9/00; A61L 2/10; C02F 2303/04; C02F 2201/3221; A61L 2202/11; A47L 11/4016

(57) ABSTRACT

A multifunctional mopping machine includes a handle, a body and a main machine. The body includes a mounting base, and a housing provided with a cleaning assembly; the body is detachably connected to the mounting base; the main machine includes a negative pressure assembly and a power supply assembly; the power supply assembly, the negative pressure assembly and the cleaning assembly form a cleaning mechanism; and a water tank for supplying water to the cleaning assembly, and a sodium hypochlorite electrolysis generator are arranged on the mounting base. An ultraviolet sterilizing lamp facing the ground to be cleaned is arranged on the housing. A tail end of a dust collecting hose communicates with a negative pressure end of the main machine, and collects both dirty water and dust on the floor, so as to ensure no stain left on the floor, satisfying present floor washing requirements.

### CROSS REFERENCE TO RELATED APPLICATIONS

The present application claims the benefit of Chinese Patent Application No. 202021585797.3 filed on Aug. 3, 2020, the contents of which are incorporated herein by reference in their entirety.

### FIELD OF THE INVENTION

The present invention falls within the field of household items, in particular relates to a multifunctional mopping machine which improves a cleaning and disinfection effect and can be easily operated.

### BACKGROUND OF THE INVENTION

As a ground or floor cleaning apparatus is continually updated, various cleaning apparatuses such as a semi-automatic sweeper and mopping machine appear, and would be an essential cleaning assistant for every family as a common home appliance in the near future. The product would also develop from primary intelligence to higher-level intelligence, gradually replacing manual cleaning. As the domestic living standard in China continually increases, the mopping machine that has long been sold in the European and American market becomes available for common people, and is accepted by more and more people. A mopping cloth of a sweeper is very dirty, containing a lot of debris and bacteria. If the mopping cloth is not sterilized, the subsequent cleaning work would be conducted in the case that there is a lot of bacteria in water, which affects a cleaning effect and the health of a user.

### SUMMARY OF THE INVENTION

The main objective of the present invention is to provide a multifunctional mopping machine which improves a cleaning and disinfection effect and can be easily operated.

The technical solution adopted by the present invention is as below: a multifunctional mopping machine, comprising a handle, a body and a main machine, wherein the body comprises a mounting base, and a housing provided with a cleaning assembly; the body is detachably connected to the mounting base; the lower end of the handle is connected to the mounting base; the main machine comprises a negative pressure assembly and a power supply assembly; the power supply assembly, the negative pressure assembly and the cleaning assembly form a cleaning mechanism; and a water tank for supplying water to the cleaning assembly, and a sodium hypochlorite electrolysis generator are arranged on the mounting base.

As improvement, an ultraviolet lamp is arranged in the housing, and the ultraviolet lamp faces the ground to be cleaned; and the multifunctional mopping machine further comprises a charging base having a self-cleaning starting program, with the body being arranged on the charging base.

Preferably, the handle comprises a hollow rod, a grip and a pulling rod, wherein the grip is assembled at the upper end of the hollow rod; the lower end of the hollow rod is rotatably assembled on the mounting base; a stop wheel with stop teeth is arranged on the mounting base at a position where the lower end of the hollow rod is rotated; the pulling rod is arranged in the hollow rod in an axially movable manner; the upper end of the pulling rod extends out of the upper port of the hollow rod; and the lower end of the pulling rod is provided with a stop notch fitted to the stop teeth.

Preferably, the pulling rod is axially connected to a reset mechanism for driving the upwards resetting of the pulling rod.

Preferably, the reset mechanism is a coil spring, the coil spring is sheathed on the pulling rod, and the coil spring is compressed between the pulling rod and the upper port of the hollow rod.

Preferably, a pulling ring is arranged at the upper end of the pulling rod.

Preferably, the hollow rod and the grip are integrally formed.

Preferably, the cleaning assembly refers to a roller brushing cylinder arranged in the front end of the housing, a dust collecting hose communicating with a dust collecting channel in the middle of the housing and extending to the roller brushing cylinder is internally mounted at the rear of the housing, the water tank is located above the dust collecting channel, at least one water discharge switching assembly is arranged at the bottom of the water tank, several water spraying pipes are mounted on a side wall inside the housing that is close to the roller brushing cylinder, the water spraying pipes and the water discharge switching assembly are connected to a water pump electric motor inside the body by means of water pipes, a dust collecting cavity with the lower end thereof opening is arranged on an upper side in the front end of the housing, a porous filtering portion is arranged between the dust collecting cavity and the dust collecting channel, and on an inner side of the dust collecting cavity in the middle, and the upper side of the dust collecting cavity is covered by means of a detachable cover plate.

Preferably, a driving electric motor is mounted at a position inside the housing that is close to the roller brushing cylinder, the driving electric motor drives the rotation of the roller brushing cylinder by means of a transmission assembly, and at least one mopping cloth is mounted at the bottom of the housing.

Preferably, a dirty water sink is also mounted on the mounting base, a tail end of the dust collecting hose communicates with the dirty water sink, and a waste water gathering area is provided in the dirty water sink. A top cover is arranged at the top of the dirty water sink, the top cover extends towards the interior of the dirty water sink to form a filtering barrel, a multilayer filter is also arranged outside the top cover, the filtering barrel communicates with the filter, and the filter communicates with the negative assembly of the main machine.

The technical effects of the present invention are: 1. an ultraviolet sterilizing lamp facing the ground to be cleaned is arranged on a housing, killing most bacteria on the ground and in dirty water, so as to ensure a clean and sterile effect; 2. a water tank capable of generating sodium hypochlorite (generated by means of electrolysis) is provided, and water sprayed thereby has a disinfection function, greatly improving a disinfection effect for the cleaned ground; 3. a tail end of a dust collecting hose communicates with a negative pressure end of a main machine, and collects both dirty water and dust on the floor, so as to ensure no stain left on the floor, satisfying present floor washing requirements; 4. a handle can be subjected to angle adjustment according to requirements, so as to adapt to people of different heights and adapt to different environment to be cleaned; 5. the main machine is detachable connected to a body, and the main machine can be separately taken off to be used as a dust collector; and 6. the present invention has an automatic cleaning function in a charged state.

### BRIEF DESCRIPTION OF THE DRAWINGS

FIG. 1 is a schematic diagram of the present invention.

FIG. 2 is a cross-sectional view of the present invention.

FIG. 3 is a schematic diagram of the present invention after the handle is rotated.

In the figures: handle 1; mounting base 2; housing 3; main machine 4; water tank 5; hollow rod 6; grip 7; a pulling rod 8; a pulling ring 8.1; a stop wheel 9; a coil spring 10; a dust collecting channel 11; a dirty water sink 12; a dust collecting hose 13; a filtering barrel 14; and a filter 15.

### DETAILED DESCRIPTION OF THE EMBODIMENTS

The following description is used for disclosing the present invention so that a person skilled in the art can realize the present invention. Preferred embodiments in the following description are merely examples, and other obvious modifications would have been conceivable for a person skilled in the art.

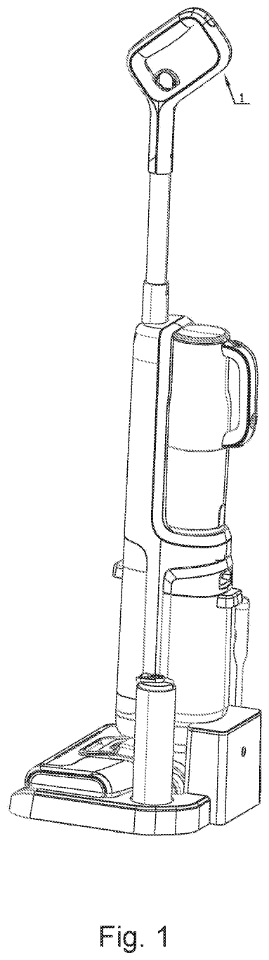

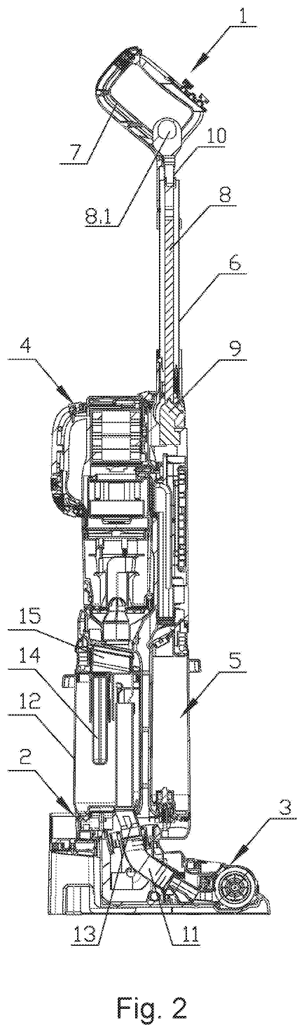

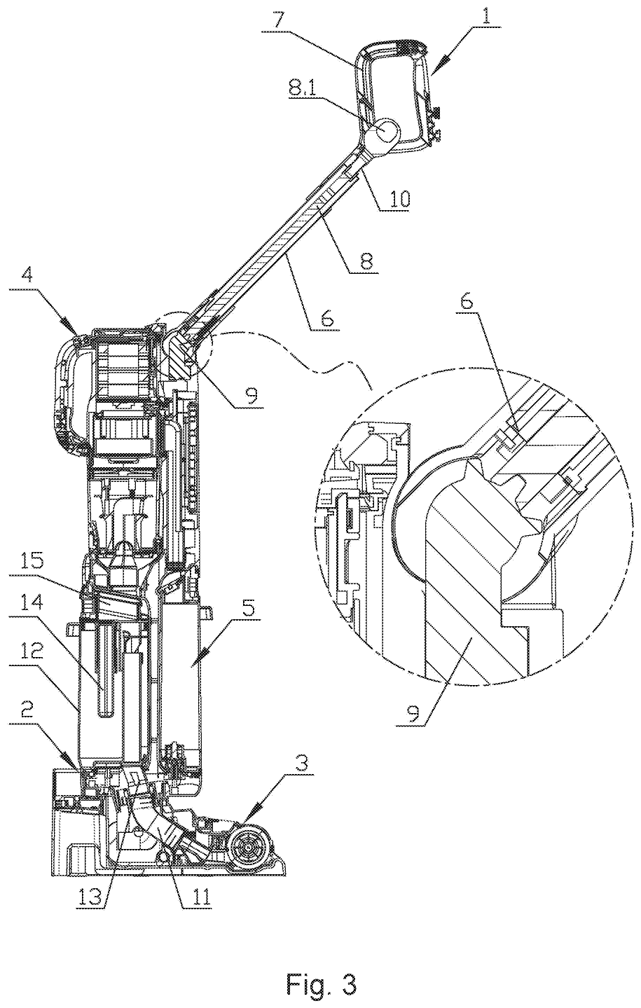

As shown in conjunction with FIG. 1 to FIG. 3, embodiment 1 specifies: a multifunctional mopping machine, comprising a handle 1, a body and a main machine 4, wherein the body comprises a mounting base 2, and a housing 3 provided with a cleaning assembly; the body 4 is detachably connected to the mounting base 2; the lower end of the handle 1 is connected to the mounting base 2; the main machine 4 comprises a negative pressure assembly and a power supply assembly; the power supply assembly, the negative pressure assembly and the cleaning assembly form a cleaning mechanism; the power supply assembly is a storage battery; the structural principle of the negative pressure assembly is the same as that of an existing dust collector; and a water tank 5 for supplying water to the cleaning assembly, and a sodium hypochlorite electrolysis generator are arranged on the mounting base 2. The sodium hypochlorite electrolysis generator is the same as that in the prior art, and mainly comprises an electrolysis device, in cooperation with saturated salt water. The saturated salt water is filled in the water tank, and sodium hypochlorite is generated by means of electrolysis.

In the present embodiment, an ultraviolet lamp is arranged in the housing 3, and the ultraviolet lamp faces the ground to be cleaned, having a disinfection function. Cleaning water can also be disinfected by means of the ultraviolet lamp. The multifunctional mopping machine further comprises a charging base having a self-cleaning starting program, with the body being arranged on the charging base. According to the setting of the program, during a charging process of the present invention, an automatic cleaning function is realized, that is, in the charged state, the self-cleaning function is automatically started, such that water spraying, the cleaning of the roller brushing cylinder, and waste water collection are realized in suit.

In the present embodiment, the handle 1 comprises a hollow rod 6, a grip 7 and a pulling rod 8, wherein the grip 7 is assembled at the upper end of the hollow rod 6; the lower end of the hollow rod 6 is rotatably assembled on the mounting base 2; a stop wheel 9 with stop teeth is arranged on the mounting base 2 at a position where the lower end of the hollow rod 6 is rotated; the pulling rod 8 is arranged in the hollow rod 6 in an axially movable manner; the upper end of the pulling rod 8 extends out of the upper port of the hollow rod 6; and the lower end of the pulling rod 8 is provided with a stop notch fitted to the stop teeth. When the pulling rod 8 is pulled up and the lower end of the pulling rod 8 is disengaged from a stop tooth on the stop wheel 9, the hollow rod 6 is rotated. After a required angle is reached, the pulling rod 8 is released. The pulling rod 8 is axially connected to the reset mechanism for driving the upwards resetting of the pulling rod 8. Under the action of the resetting mechanism, the pulling rod 8 is reset, and the lower end of the pulling rod 8 is fitted to another stop tooth on the stop wheel 9 to realize limitation, ensuring the angle of the hollow rod 6 after being rotated.

In the present embodiment, the reset mechanism is a coil spring 10, the coil spring 10 is sheathed on the pulling rod 8, and the coil spring 10 is compressed between the pulling rod 8 and the upper port of the hollow rod 6. The pulling rod 8 is provided, below the lower end of the coil spring 10, with a spring base that is fixed to an outer wall of the pulling rod 8, realizing the axial limitation of the lower end of the coil spring 10. The upper end of the coil spring 10 is limited below the upper port of the hollow rod 6, that is, the upper port of the hollow rod 6 forms a radial inwards extension, and the upper end of the coil spring 10 is radially limited at a lower surface of the radial inwards extension.

In the present embodiment, a pulling ring 8.1 is arranged at the upper end of the pulling rod 8, thereby facilitating the pulling of the pulling rod 8 by means of the pulling ring 8.1.

In the present embodiment, the hollow rod 6 and the grip 7 are integrally injection molded or casting formed.

In the present embodiment, the cleaning assembly refers to a roller brushing cylinder arranged in the front end of the housing 3, a dust collecting hose 13 communicating with a dust collecting channel 11 in the middle of the housing 3 and extending to the roller brushing cylinder is internally mounted at the rear of the housing 3, the water tank 5 is located above the dust collecting channel 11, at least one water discharge switching assembly is arranged at the bottom of the water tank 5, several water spraying pipes are mounted on a side wall inside the housing 3 that is close to the roller brushing cylinder, the water spraying pipes and the water discharge switching assembly are connected to a water pump electric motor inside the body by means of water pipes, a dust collecting cavity with the lower end thereof opening is arranged on an upper side in the front end of the housing 3, a porous filtering portion is arranged between the dust collecting cavity and the dust collecting channel 11, and on an inner side of the dust collecting cavity in the middle, and the upper side of the dust collecting cavity is covered by means of a detachable cover plate.

In the present embodiment, a driving electric motor is mounted at a position inside the housing 3 that is close to the roller brushing cylinder, the driving electric motor drives the rotation of the roller brushing cylinder by means of a transmission assembly, and at least one mopping cloth is mounted at the bottom of the housing 3.

In the present embodiment, a dirty water sink 12 is also mounted on the mounting base 2, a tail end of the dust collecting hose 13 communicates with the dirty water sink 12, and a waste water gathering area is provided in the dirty water sink 12. A top cover is arranged at the top of the dirty water sink 12, the top cover extends towards the interior of the dirty water sink 12 to form a filtering barrel 14, a multilayer filter 15 is also arranged outside the top cover, the filtering barrel 14 communicates with the filter 15, and the filter 15 communicates with the negative assembly of the main machine 4.

1. A multifunctional mopping machine, comprising a handle (1), a body and a main machine (4), wherein the body comprises a mounting base (2), and a housing (3) provided with a cleaning assembly; the body (4) is detachably connected to the mounting base (2); a lower end of the handle (1) is connected to the mounting base (2); the main machine (4) comprises a negative pressure assembly and a power supply assembly; the power supply assembly, the negative pressure assembly and the cleaning assembly form a cleaning mechanism; and a water tank (5) for supplying water to the cleaning assembly, and a sodium hypochlorite electrolysis generator are arranged on the mounting base (2).

2. The multifunctional mopping machine according to claim 1, wherein an ultraviolet lamp is arranged in the housing (3), and the ultraviolet lamp faces a ground to be cleaned; and the multifunctional mopping machine further comprises a charging base having a self-cleaning starting program, with the body being arranged on the charging base.

3. The multifunctional mopping machine according to claim 1, wherein the handle (1) comprises a hollow rod (6), a grip (7) and a pulling rod (8), wherein the grip (7) is assembled at an upper end of the hollow rod (6); the lower end of the hollow rod (6) is rotatably assembled on the mounting base (2); a stop wheel (9) with stop teeth is arranged on the mounting base (2) at a position where the lower end of the hollow rod (6) is rotated; the pulling rod (8) is arranged in the hollow rod (6) in an axially movable manner; the upper end of the pulling rod (8) extends out of an upper port of the hollow rod (6); and the lower end of the pulling rod (8) is provided with a stop notch fitted to the stop teeth.

4. The multifunctional mopping machine according to claim 3, wherein the pulling rod (8) is axially connected to a reset mechanism for driving upwards resetting of the pulling rod (8).

5. The multifunctional mopping machine according to claim 4, wherein the reset mechanism is a coil spring (10), the coil spring (10) is sheathed on the pulling rod (8), and the coil spring (10) is compressed between the pulling rod (8) and the upper port of the hollow rod (6).

6. The multifunctional mopping machine according to claim 3, wherein a pulling ring (8.1) is arranged at the upper end of the pulling rod (8).

7. The multifunctional mopping machine according to claim 4, wherein a pulling ring (8.1) is arranged at the upper end of the pulling rod (8).

8. The multifunctional mopping machine according to claim 5, wherein a pulling ring (8.1) is arranged at the upper end of the pulling rod (8).

9. The multifunctional mopping machine according to claim 3, wherein the hollow rod (6) and the grip (7) are integrally formed.

10. The multifunctional mopping machine according to claim 1, wherein the cleaning assembly refers to a roller brushing cylinder arranged in a front end of the housing, a dust collecting hose (13) communicating with a dust collecting channel (11) in a middle of the housing (3) and extending to the roller brushing cylinder is internally mounted at a rear of the housing (3), the water tank (5) is located above the dust collecting channel (11), at least one water discharge switching assembly is arranged at a bottom of the water tank (5), several water spraying pipes are mounted on a side wall inside the housing (3) that is close to the roller brushing cylinder, the water spraying pipes and the water discharge switching assembly are connected to a water pump electric motor inside the body by means of water pipes, a dust collecting cavity with a lower end thereof opening is arranged on an upper side in the front end of the housing (3), a porous filtering portion is arranged between the dust collecting cavity and the dust collecting channel (11), and on an inner side of the dust collecting cavity in the middle, and the upper side of the dust collecting cavity is covered by means of a detachable cover plate.

11. The multifunctional mopping machine according to claim 10, wherein a driving electric motor is mounted at a position inside the housing (3) that is close to the roller brushing cylinder, the driving electric motor drives a rotation of the roller brushing cylinder by means of a transmission assembly, and at least one mopping cloth is mounted at the bottom of the housing (3).

12. The multifunctional mopping machine according to claim 10, wherein a dirty water sink (12) is also mounted on the mounting base (2), a tail end of the dust collecting hose (13) communicates with the dirty water sink (12), and a waste water gathering area is provided in the dirty water sink (12); a top cover is arranged at a top of the dirty water sink (12), the top cover extends towards an interior of the dirty water sink (12) to form a filtering barrel (14), a multilayer filter (15) is also arranged outside the top cover, the filtering barrel (14) communicates with the multilayer filter (15), and the multilayer filter (15) communicates with the negative assembly of the main machine (4).
